(19) United States
(12) Patent Application Publication
(10) Pub. No.: US 2016/0220465 A1
(43) Pub. Date: Aug. 4, 2016

(54) MINOXIDIL FORMULATIONS

(71) Applicant: APPLIED BIOLOGY, INC., Irvine, CA (US)

(72) Inventors: Ofer A. GOREN, Newport Beach, CA (US); John MCCOY, Downey, CA (US)

(73) Assignee: APPLIED BIOLOGY, INC., Irvine, CA (US)

(21) Appl. No.: 14/986,071

(22) Filed: Dec. 31, 2015

Related U.S. Application Data
(60) Provisional application No. 62/109,288, filed on Jan. 29, 2015.

(51) Int. Cl. A61K 8/49; A61Q 5/02; A61Q 7/00; A61K 8/36; A61K 8/81; A61K 8/04; A61K 8/73

(52) U.S. Cl. CPC A61K 8/4953; A61K 8/046; A61Q 5/02; A61K 8/732; A61K 8/361; A61K 8/8141; A61K 8/36; A61Q 7/00

(57) ABSTRACT

Disclosed herein are cosmetically pleasing topic al formulations of minoxidil and methods of inducing hair growth. The minoxidil is at a concentration of 2%-20% by weight in the formulations. In one embodiment, the aerosol formulation comprises a sebum absorber such as a modified starch.

### TECHNICAL FIELD

The present invention relates to topical compositions for preventing, re-growing, and treating hair loss.

### BACKGROUND

Topical minoxidil (2% solution, 5% solution, and 5% aerosol, foam) are the only FDA approved drugs indicated for the treatment of female and male pattern hair loss. Even though it is estimated that up to 50% of men and women will suffer from pattern hair loss by the age of 65, topical minoxidil has had a limited success in the market primary due to poor compliance.

A survey of dermatologists as well as female pattern hair loss patients attributed the lack of compliance to the greasy feel and look of current commercially available topical minoxidil formulations and their effect on hair and disruption of hair style. The technology described herein provides cosmetically pleasing formulations of minoxidil for topical application that leave little or no greasy residue on the hair and minimally disrupts patients' hair styles.

One embodiment of the invention disclosed herein provides an aerosol composition that applies a minoxidil dry shampoo containing a sebum absorbing compound such as a modified starch, which itself is not greasy and additionally will remove the greasy particulate from the hair and thus increase compliance.

### SUMMARY

The inventions herein relate to formulations of minoxidil or a pharmaceutically acceptable salt thereof formulated (1) as an aerosol for application not as a foam or mousse, (2) as an aerosol for application as a foam; (3) as a liquid solution, or (4) as a gel.

The formulation of type (1) is an aerosol composition comprising a dry shampoo and a propellant, wherein the dry shampoo comprises minoxidil or a pharmaceutically acceptable salt thereof at a concentration of about 2 to about 20% by weight; a carrier material; and a modified starch such as aluminum starch octenyl succinate; wherein the composition is not formulated for application as a foam or mousse. The formulation of type (2) is a foam composition comprising minoxidil or a pharmaceutically acceptable salt thereof at a concentration of about 2 to about 20% by weight; a modified starch such as aluminum starch octenyl succinate; and a propellant. The formulation of type (3) is a liquid composition comprising minoxidil or a pharmaceutically acceptable salt thereof at a concentration of about 2 to about 20% by weight; aluminum starch octenyl succinate at a concentration of from 2% to 20% by weight; and a pharmaceutically acceptable solvent. The formulation of type (4) is a gel composition comprising minoxidil or a pharmaceutically acceptable salt thereof at a concentration of about 2 to about 20% by weight; aluminum starch octenyl succinate at a concentration of from 2% to 20% by weight; a gelling agent; and a pharmaceutically acceptable solvent. The formulations described herein may be used in methods of inducing hair growth or reducing the rate of hair loss of a subject in need thereof, comprising applying a therapeutically effective amount of the formulation at least once daily to the scalp of the subject.

### DETAILED DESCRIPTION

The technology described herein addresses the deficiencies in the currently available topical minoxidil products in order to increase patients' compliance. In one aspect, the technology described herein is related to an aerosol formulation (not a foam or mousse) that contains ingredients that facilitate a more pleasing aesthetic, for example, a sebum absorber such as a modified starch that can absorb sebum from the scalp and hair. Such compositions may reduce or eliminate an oily and/or unaesthetic appearance of hair as well as avoid the greasy characteristics found in many commercially available minoxidil formulations.

Aerosol, Non-Foaming Compositions

In one embodiment, the formulation disclosed herein is an aerosol composition comprising a dry shampoo and a propellant, wherein the composition is not formulated for application as a foam or mousse. By “dry shampoo” is meant a formulation comprising a carrier material that is a volatile liquid and therefore evaporates and a powder that remains, wherein the powder contains a starch or modified starch, such as aluminum starch octenyl succinate. In the present formulations, the dry shampoo comprises minoxidil, a carrier material and a modified starch. Examples of suitable carrier materials that are volatile liquids are lower alcohols including without limitation ethanol or isopropanol, a volatile silicone compound such as polydimethylsiloxanes (e.g., having a viscosity less than about 5 cSt at 25° C.), cyclomethicone, cyclohexane siloxane, decamethyltetrasiloxane, octamethyltrisiloxane, decamethylpentasiloxane, decamethylcyclopentasiloxane, octamethylcyclotetrasiloxane, trimethylsilylamodimethicone, phenyl trimethicone, hexamethyidisiloxane, and dimethylsiloxane/methylalkylsiloxane, and combinations thereof. Other carrier materials known to those skilled in the art may also be used. The total percentage weight of the carrier material in the aerosol composition may be between about 0.1% and about 50%, between about 0.1% and about 40%, between about 1% and about 35%, between about 5% and about 50%, between about 10% and about 40%, between about 15% and about 40%, of the total weight of the aerosol dry shampoo composition (a combination of the dry shampoo composition and the propellant). In certain embodiments, the composition is substantially free of water.

In one embodiment, the dry shampoo further comprises a solvent, which may or may not be volatile. The solvent can be an alcohol, such as a polyhydric alcohol. Non-limiting examples of polyhydric alcohols include 1,3-butylene glycol, propylene glycol, glycol 200 (PEG 200), polyethylene glyco1400 (PEG 400), hexylene glycol and dipropylene glycol, and glycerol. The polyhydric alcohol can have a concentration of 10% or less by weight, or 5% or less by weight, or between 10% to 1% by weight. In one embodiment, the solvent can comprise benzyl alcohol. In another embodiment, the solvent comprises propylene glycol and/or water. Low levels of water (such as 1-5% or less than 10%) are preferred in the non-foaming aerosol compositions of the invention. If the solvent is non-volatile, low levels of less than 20%, less than 10%, less than 5% or less than 1% of the total weight of the aerosol dry shampoo composition (a combination of the dry shampoo composition and the propellant) is preferred.

The dry shampoo in the present embodiments further comprises a starch or modified starch. The starch or modified starch preferably acts as a sebum absorber. Non-limiting examples of suitable starch materials include cornstarch, potato starch, tapioca starch, rice starch, wheat starch, and cassaya starch. A starch material may be modified or unmodified. A modified starch material is a starch which has been derivatized or altered by processes known to those of ordinary skill in the art, such as esterification, etherification, oxidation, acid hydrolysis, crosslinking, or enzyme conversion. Non-limiting examples of suitable modified starch materials include aluminum starch octenylsuccinate, sodium starch octenylsuccinate, calcium starch octenylsuccinate, distarch phosphate, hydroxyethyl starch phosphate, hydroxypropyl starch phosphate, sodium carboxymethyl starch, and sodium starch glycolate. In an embodiment, the dry shampoo comprises aluminum starch octenyl succinate and tapioca starch.

In some embodiments the dry shampoo further comprises another oil-absorbing powder, for example, cellulose, chalk, talc, fuller's earth, etc. In some embodiments, a further sebum absorber is added, including a clay material, which may be stearalkonium hectorite. In some embodiments, the further sebum absorber is at least one modified clay material selected from the group consisting of stearalkonium hectorite, stearalkonium bentonite, quaternium-18 bentonite, and quaternium-18 hectorite. In some embodiments, silica is added and may function as an oil-absorbing compound and/or a suspending agent. In another embodiment, the composition is substantially free of silica and silica-containing components.

The starch material may be present in the dry shampoo at a concentration of 1% to 70% by weight, 1% to 60% by weight, 1% to 50% by weight, 1% to 40% by weight, 1% to 30% by weight, 1% to 20% by weight, 1% to 15% b y weight, 1% to 10% by weight, 5% to 50% by weight, 5% to 40% by weight, 5% to 30% by weight, 5% to 20% by weight, 5% to 10% by weight, 5% to 15% by weight, 10% to 60% by weight, 10% to 50% by weight, 10% to 40% by weight, 10% to 30% by weight, 10% to 20% by weight, 10% to 15% by weight, or 20% to 60% by weight as measured based on the total weight of the dry shampoo.

In certain embodiments, the aerosol composition comprises a dry shampoo having as a starch or modified starch aluminum starch octenylsuccinate at a concentration in the dry shampoo of 2% to 20% by weight, 2% to 10% by weight, 10% to 20% by weight, 11% to 20% by weight, 10% to 15% by weight, 14 to 16% by weight, 2% to 10% by weight, 2 to 3% by weight, 3% to 4% by weight, 5% to 6% by weight, 7% to 8% by weight, 9% to 10% by weight, 11% to 12% by weight, 13% to 14% by weight, 13% to 15% by weight, 15% to 16% by weight, 17% to 18% by weight, 13% to 16% by weight, 14% to 16% by weight, 19% to 20% by weight, 2 to 8% by weight, 2 to 5% by weight, 4 to 8% by weight, or 5 to 10% by weight, or at a concentration of about 5%, 6%, 7%, 8%, 9%, 10%, 11%, 12%, 13%, 14%, 15%, 16%, 17%, 18%, 19%, or 20% by weight.

The aerosol compositions described herein also include a propellant. Non-limiting examples of suitable propellants include butane, isobutane, propane, A-46 (isobutane and propane), liquefied petroleum gas (e.g., propane), dimethyl ether, methyl ethyl ether, trichlorofluoromethane, dichlorodifluoromethane, dichlorotetrafluorothane, monochlorodifluoromethane, trichlorotrifluoroethane propane, carbon dioxide, nitrous oxide, 1,1,1,2,-tetrafluoroethane, 1,1,2,3,3,3-heptafluoropropane, or combinations thereof. In a particular embodiment, the propellant is isobutane. A propellant may condense to a liquid state in an aerosol container at ambient temperatures. In some embodiments, the propellant may have a lower specific gravity as compared to the rest of the composition, thus facilitating propelling the composition from a container (e.g., through a dip tube) as compared to expelling the propellant.

The propellant is present at a concentration of 25% to 90% by weight, 25% to 80% by weight, 25% to 70% by weight, 25% to 60% by weight, 25% to 50% by weight, 25% to 40% by weight, 25% to 30% by weight, 30% to 90% by weight, 30% to 80% by weight, 30% to 70% by weight, 30% to 60% by weight, 30% to 50% by weight, 30% to 40% by weight, 40% to 90% by weight, 40% to 80% by weight, 40% to 70% by weight, 40% to 60% by weight, 40% to 50% by weight, 50% to 90% by weight, 50% to 80% by weight, 50% to 70% by weight, or 50% to 60% by weight of the total composition (dry shampoo and propellant).

Examples of Aerosol, Non-Foaming Compositions

As an example of one particular aerosol composition according to the present invention (nonfoaming) is an aerosol composition comprising about 2% to about 20% by weight of minoxidil or a pharmaceutically acceptable salt thereof, about 2% to about 20%, or about 2% to about 10% by weight of aluminum starch octenylsuccinate. This composition may further comprise alcohol, propylene glycol, and water. A second example of an aerosol composition according to the present invention (nonfoaming) is an aerosol composition comprising a propellant and a dry shampoo, wherein the dry shampoo comprises aluminum starch octenyl succinate at a concentration of 2%-10% by weight, minoxidil or a pharmaceutically acceptable salt thereof at a concentration of 2%-15% by weight, SD alcohol 40-B, and polysorbate 60. A third example is an aerosol composition comprising a propellant and a dry shampoo, wherein the dry shampoo comprises 2%-20% by weight of aluminum starch octenyl succinate, about 6% to about 15% by weight of minoxidil or a pharmaceutically acceptable salt thereof, ethanol, and an acid, wherein the composition has an apparent pH between 5.0 and 7.0. In this third embodiment, the dry shampoo may further comprise about 1%-5% by weight of magnesium stearate. In a fourth example, the aerosol composition comprises a propellant and a dry shampoo, wherein the dry shampoo comprises 2%-20% by weight of aluminum starch octenyl succinate, about 6% to about 15% by weight of minoxidil or a pharmaceutically acceptable salt thereof, ethanol, an acid, acrylates copolymer and magnesium carbonate, wherein the combined concentration of acrylates copolymer and magnesium carbonate is 5%-15% by weight. This embodiment further comprises a propellant containing dimethyl ether. In another embodiment, the dry shampoo comprises 10%-17% by weight of aluminum starch octenyl succinate, about 6% to about 15% by weight of minoxidil or a pharmaceutically acceptable salt thereof, ethanol, an acid at an apparent pH between 6.0 and 6.5, and 5%-15% by weight of a combination of acrylates copolymer and magnesium carbonate. This embodiment may further comprise about 1%-5% by weight of magnesium stearate. In a further preferred embodiment, the dry shampoo comprises about 14% by weight of aluminum starch octenyl succinate, about 6% to about 15% by weight of minoxidil or a pharmaceutically acceptable salt thereof, ethanol, lactic acid at an apparent pH between 6.0 and 6.5, and 5-15% by weight of a combination of acrylates copolymer and magnesium carbonate.

In another embodiment, the technology described herein provides an aerosol composition comprising a propellant and a dry shampoo, wherein the dry shampoo comprises aluminum starch octenyl succinate at a concentration of 2%-20% by weight, minoxidil or a pharmaceutically acceptable salt thereof at a concentration of about 2% or about 5% by weight, ethanol, and propylene glycol. In this embodiment, the composition may be substantially free of water. It may further comprise in the dry shampoo acrylates copolymer and magnesium carbonate. A suitable propellant for this embodiment is dimethyl ether.

In another example of an aerosol composition within the present disclosure is an aerosol composition comprising a propellant and a dry shampoo, wherein the dry shampoo comprises about 13%-17% by weight of aluminum starch octenyl succinate, about 2% or about 5% by weight of minoxidil or a pharmaceutically acceptable salt thereof, ethanol, about 3%-6% by weight of magnesium stearate, and an acid at an apparent pH between 5.0 and 7.0.

In another embodiment, the dry shampoo comprises 13-15% by weight of aluminum starch octenyl succinate, 2% by weight of minoxidil or a pharmaceutically acceptable salt thereof, ethanol, an acid at an apparent pH between 6.0 and 6.5, and 5-15% by weight of a combination of acrylates copolymer and magnesium carbonate. In one embodiment, the dry shampoo further comprises about 1%-5% by weight of magnesium stearate. In yet another embodiment, the dry shampoo comprises about 16% by weight of aluminum starch octenyl succinate, about 2% by weight of minoxidil or a pharmaceutically acceptable salt thereof, ethanol, lactic acid at an apparent pH between 6.0 and 6.5, and 5-15% by weight of a combination of acrylates copolymer and magnesium carbonate. In one embodiment, the dry shampoo further comprises about 5% by weight of magnesium stearate.

In another embodiment, the technology described herein provides an aerosol composition comprising a propellant and a dry shampoo, wherein the dry shampoo comprises aluminum starch octenyl succinate at a concentration of 2%-20% by weight, tapioca starch, minoxidil or a pharmaceutically acceptable salt thereof at a concentration of 2%-20% by weight, SDA-40-2, and an acid. In this embodiment, the propellant comprises A-46 (15.2% propane, 84.8% isobutene), propane and/or isobutane.

In another embodiment, the technology described herein provides an aerosol composition comprising a propellant and a dry shampoo, wherein the dry shampoo comprises about 2%-20% or about 6% to about 20% by weight of aluminum starch octenyl succinate, about 6% to about 20% or about 6% to about 15% by weight of minoxidil or a pharmaceutically acceptable salt thereof, and a solvent. Alternatively, in this embodiment, the dry shampoo comprises about 2%-10% by weight of aluminum starch octenyl succinate. In another alternative, the dry shampoo further comprises about 2%-5% by weight of magnesium stearate, and/or is substantially free of water. In another variation of this embodiment, the dry shampoo comprises about 13%-17% by weight of aluminum starch octenyl succinate, about 6% to about 20% by weight of minoxidil or a pharmaceutically acceptable salt thereof, and a solvent.

In another embodiment, the technology described herein provides an aerosol composition comprising a propellant and a dry shampoo, wherein the dry shampoo comprises about 13%-17% by weight of aluminum starch octenyl succinate, about 2%-7% by weight tapioca starch, about 6% to about 15% by weight of minoxidil or a pharmaceutically acceptable salt thereof, SDA-40-2, and an acid at an apparent pH between 5.0 and 7.0. In one embodiment, the dry shampoo further comprises about 2%-5% by weight of magnesium stearate. In one embodiment, the composition furthermore is substantially free of water. In another embodiment, the dry shampoo comprises about 14-16% by weight of aluminum starch octenyl succinate, about 4%-6% by weight tapioca starch, about 6% to about 15% by weight of minoxidil or a pharmaceutically acceptable salt thereof, SDA-40-2, and an acid at an apparent pH between 6.0 and 6.5. In one embodiment, the dry shampoo further comprises about 2%-4% by weight of magnesium stearate. In a further preferred embodiment, the dry shampoo comprises about 15% by weight of aluminum starch octenyl succinate, about 6% by weight of tapioca starch, about 6% to about 15% by weight of minoxidil or a pharmaceutically acceptable salt thereof, SDA-40-2, and lactic acid at an apparent pH between 6.0 and 6.5.

In an embodiment, the technology described herein provides an aerosol composition comprising a propellant and a dry shampoo, wherein the dry shampoo comprises about 13%-17% by weight of aluminum starch octenyl succinate, about 2%-7% by weight tapioca starch, 5% by weight of minoxidil or a pharmaceutically acceptable salt thereof, SDA-40-2, and an acid at an apparent pH between 5.0 and 7.0. In one embodiment, the minoxidil or a pharmaceutically acceptable salt thereof is fully dissolved in the acid. In one embodiment, the acid is lactic acid. In another embodiment, the dry shampoo comprises about 14-16% by weight of aluminum starch octenyl succinate, about 4%-6% by weight tapioca starch, 5% b y weight of minoxidil or a pharmaceutically acceptable salt thereof, SDA-40-2, and an acid at an apparent pH between 6.0 and 6.5. In a further preferred embodiment, the dry shampoo comprises about 15% by weight of aluminum starch octenyl succinate, about 6% by weight of tapioca starch, 5% by weight of minoxidil or a pharmaceutically acceptable salt thereof, SDA-40-2, and lactic acid at an apparent pH between 6.0 and 6.5.

In another embodiment, the technology described herein provides an aerosol composition comprising a propellant and a dry shampoo, wherein the dry shampoo comprises 2%-20% by weight of aluminum starch octenyl succinate, about 4%-6% by weight tapioca starch, 2% by weight of minoxidil or a pharmaceutically acceptable salt thereof, SDA-40-2, and an acid at an apparent pH between 5.0 and 7.0.

In another embodiment, the technology described herein provides an aerosol composition comprising minoxidil or a pharmaceutically acceptable salt thereof at a concentration of about 2% to about 5% by weight; and a solvent comprising a lower alcohol, wherein the composition is not formulated for application as a foam or mousse. This composition may further include a modified starch, and further comprises a propellant that is present at a concentration of 25-90% by weight of the total weight of the aerosol composition. It may further comprise at least one additive component, such as polysorbate 60. Preferably, the foregoing composition is stored in a non-combustible propellant delivery system, which may be a bag-on-valve system. In another embodiment, the technology described herein provides an aerosol composition comprising a propellant, a modified starch such as aluminum starch octenylsuccinate, minoxidil or a pharmaceutically acceptable salt thereof at a concentration of about 2% or about 5% by weight, and SD alcohol 40-B, and optionally polysorbate 60 and/or lactic acid, wherein the minoxidil or a pharmaceutically acceptable salt thereof is fully dissolved in the composition, and wherein the composition is substantially free of water.

Liquid Compositions

The present disclosure also concerns a liquid composition that is administered using a spray device lacking a chemical propellant, a dropper, or otherwise mechanically spread on the scalp or skin without the use of a propellant. Such a liquid composition comprises minoxidil or a pharmaceutically acceptable salt thereof, aluminum starch octenyl succinate, and a pharmaceutically acceptable solvent. The solvent may be one readily apparent to one in the field, and may be one or more of the carrier materials and the solvents discussed above for the aerosol composition (nonfoaming). Preferably, the compositions described herein are formulated for application as a solution spray. The composition described herein can be applied directly to the hair or scalp, by using a container fitted with a pump to dispense a liquid composition (e.g., an atomizer), or by a pump aerosol container which utilizes compressed air as the propellant.

The liquid composition preferably contains aluminum starch octenyl succinate at the weight percentages discussed concerning the aerosol composition (nonfoaming). Additional adjuvants may be used in the formulation according to the disclosure herein of suitable adjuvants. A suitable solvent is water adjusted to a pH to allow for dissolution of all the composition components.

Other Compositions

Another embodiment of the invention is a foam composition comprising minoxidil at a concentration of about 2% to about 20% by weight of the composition (excluding propellant weight), a modified starch such as without limitation aluminum starch octenylsuccinate, a gelling agent, and a propellant. The foam composition may be a breakable foam that breaks upon application of shear pressure. The propellant may be any of the propellants discussed concerning the aerosol composition (nonfoaming). The gelling agent may be any of those known to one of ordinary skill in the art, such as methylcellulose, poloxamers, bentonite, gelatin, sodium carboxymethyl cellulose, carbomers, tragacanth, and alginic acid.

Another embodiment of the invention is a gel composition comprising minoxidil at a concentration of about 2% to about 20% by weight of the composition, a modified starch such as without limitation aluminum starch octenylsuccinate, a gelling agent, and a solvent. The gelling agent may be any of those known in the art including the gelling agents discussed herein concerning the foam composition. The solvent may be any suitable for dissolving or suspending minoxidil and the gelling agent.

Minoxidil Concentration

The minoxidil or a pharmaceutically acceptable salt thereof is present in the compositions disclosed herein at a concentration of about 2% to about 20% by weight of the total composition (excluding propellant for the aerosol formulations). In certain embodiments, the minoxidil or salt thereof has a concentration of about 2, 3, 4, 5, 6, 7, 8, 9, 10, 11, 12, 13, 14, 15, 16, 17, 18, 19, or 20% by weight of the total composition (excluding propellant for aerosol formulations). In other embodiments, the minoxidil or a pharmaceutically acceptable salt thereof has a concentration of 1-3%, 2-5%, 5-10%, 4-6%, 9-11%, 10-15%, 14-16%, 15-20%, or 19-21% by weight of the total composition (excluding propellant for aerosol formulations). Suitable minoxidil salts may be any of those known to one of skill in the art. Suitable salts include without limitation acetate, citrate, succinate, benzoate, hydrochloride, sulphate, phosphate, or lactate. In certain embodiments, the minoxidil salt is a minoxidil acetate, succinate, lactate, or citrate salt.

It was previously disclosed in U.S. Pat. No. 6,946,120 (Wai-Chiu So et al.) that the solubility of minoxidil or a pharmaceutically acceptable salt thereof can be significantly increased by adjusting the acid concentration in the composition. The compositions described in U.S. Pat. 6,946,120 are formulated for application as a foam or mousse. While not required in some embodiments, such as those with 2%-5% by weight of minoxidil or a pharmaceutically acceptable salt thereof, other embodiments may benefit from the addition of an acid, particularly compositions having higher concentrations such as 5% to 20% by weight of minoxidil. The acid may increase the solubility of minoxidil. Accordingly, any of the embodiments describe herein, and particularly the embodiments containing greater than 5% by weight of minoxidil or a pharmaceutically acceptable salt thereof, may further comprise an acid, and preferably one of the acids described herein.

In one embodiment, the acid is added in an amount sufficient to provide an apparent pH to the composition of approximately 7.0 or less. The apparent pH of the composition can be between 5.0 to 7.0 or between 6.0 to 6.5. Any suitable acid can be used to adjust the pH, including mineral acids, such as hydrochloric acid, sulphuric acid, nitric acid and phosphoric acid, or organic acids such as citric acid, acetic acid, lactic acid, succinic acid, or maleic acid, or mixtures thereof. In one embodiment, the acid is acetic acid or lactic acid.

Methods of Use

The compositions described herein can be used for topical application to the scalp and/or hair. Suitable dosages are apparent from the art and routine experimentation. Patients who are not responsive to formulations with about 2% or about 5% by weight of minoxidil or a pharmaceutically acceptable salt thereof may be responsive to a higher dosage. Accordingly, a formulation including about 6% to about 20% by weight of minoxidil or a pharmaceutically acceptable salt thereof may be required for these patients. In some embodiments, the formulation may include about 6% to about 15% by weight of minoxidil or a pharmaceutically acceptable salt thereof, which may be administered to patients not showing acceptable hair regrowth or prevention of hair loss at lower minoxidil concentrations.

In one embodiment, the composition described herein is not washed off as part of the application process.

The invention is directed to a method of inducing hair growth and/or retarding or reducing hair loss by applying a minoxidil composition disclosed herein at least once daily to a scalp of a subject. The method may be considered as having a non-therapeutic cosmetic purpose.

To utilize the compositions described herein, a user or patient can apply an effective amount of the composition to the scalp. The term “effective amount,” as used herein, is an amount which is effective in preventing or treating hair loss. The user or patient is a human. The compositions described herein can be used for topical application to the scalp and/or skin. Embodiments herein are contemplated for use with men and women. In some embodiments, the user or patient is suffering from hair loss that may be related to any of the forms of alopecia including male or female pattern alopecia. Related indications may include weakening of hair strength, thinning of hair, loss of hair color and the like.

The minoxidil compositions described herein can be used once a day or multiple times a day such as twice a day, three times a day, or four times a day. In one embodiment, the composition is administered to a subject once, twice, three times, or four times every other day (i.e., on alternate days), once, twice, three times, or four times every two days, once, twice, three times, or four times every three days, once, twice, three times, or four times every four days, once, twice, three times, or four times every 5 days, once, twice, three times, or four times a week, once, twice, three times, or four times every two weeks. Preferably, the composition is administered to a subject once a day.

The dosage of the dispensed minoxidil can be controlled by counting the time of continuous spray application to the treatment area. In one embodiment, the minoxidil is administered in a metered dose spray device such that the dosage may be measured by the number of times a button or valve is pushed or released.

In some embodiments, the compositions described herein are provided as a kit. A “kit” typically defines a package including at least one composition of the invention and instructions. “Instructions” may define a component of promotion, and typically involve written instructions on or associated with packaging of compositions of the invention. Instructions also can include any oral or electronic instructions provided in any manner. A kit may also be a package containing at least one composition of the invention and a device for administering the composition, such as a spray container or a dropper.

Pharmaceutically Acceptable Carriers and Adjuvants

In one embodiment, the minoxidil can be encapsulated in a pharmaceutically acceptable carrier. The pharmaceutically acceptable carrier can encapsulate the minoxidil in liposomes, microbeads, etc. In addition to being “pharmaceutically acceptable,” each carrier must also be “acceptable” in the sense of being compatible with the other ingredients of the formulation. In one embodiment, the minoxidil is encapsulated in liposomes.

The compositions of the invention may further comprise an adjuvant such as a skin penetrating agent. Suitable skin penetrating agents include alcohols such as dodecanol and oleyl alcohol; amines, such as isopropyl amine, diisopropyl amine, triethyl amine, triethanol amine, diisopropanolamine and ethylene diamine; carboxylic acids, such as oleic acid, linoleic acid and linolenic acid; esters, such as dibutyl sebacate, dibutyl phthalate, butyl benzoate and ethyl caprate; and others, such as Azone, N methyl pyrollidone, bile salts and urea.

The composition can further comprise one or more further adjuvants (e.g., 1, 2, 3, 4, 5, 6, or more), within a range which does not impair the effect of the present invention. If more than one adjuvant is comprised in the aerosol-formulated composition, the adjuvants may or may not have the same function(s) or characteristics. Non-limiting examples of adjuvants include: antioxidants; essential oils; perfumes; waxes; fillers; hair-fixative polymers; deodorizing agents; pediculicides; anti-dandruff agents; cosmetic and/or dermatological active agents including emollients, moisturizers, vitamins (e.g., vitamin B complexes (e.g., including thiamine, nicotinic acid, biotin, pantothenic acid, choline, riboflavin, vitamin B6, vitamin B12, pyridoxine, inositol, camitine), vitamins A, C, D, E, K and their derivatives (e.g., vitamin A palmitate) or pro-vitamins), essential fatty acids, sunscreens, herb and/or plant extracts (e.g., aloe); dispersing or suspending agents (e.g., silica); pharmaceutically active agents (e.g., poly(2-hydroxystearic acid); anti-static agents (e.g., tricetyl methyl ammonium chloride); pearlescent aids (e.g., such as coated mica, ethylene glycol distearate); opacifiers (e.g., tin); preserving agents (e.g., 1,2-dibromo-2,4-dicyano butane, benzyl alcohol, methylparaben, propyl paraben, imidazolidinyl urea); coloring agents or dyes; odor neutralizers; and sequestering agents; or combinations thereof. In some embodiments, the adjuvants may be capable of absorbing oils and/or may aid as a suspending or dispersing agent. In other embodiments, the adjuvants may provide a cooling effect to the subject when the composition is applied (e.g., menthol). Other suitable adjuvants will be known to those of ordinary skill in the art. In one embodiment, the adjuvant is polysorbate 60. In another embodiment, the adjuvant is magnesium stearate. In another embodiment, the adjuvant includes acrylates copolymer. In another embodiment, the adjuvant includes magnesium carbonate. In another embodiment, the adjuvant includes acrylates copolymer and magnesium carbonate.

Aerosol Containers and Foam Containers

The compositions described herein can be prepared using any suitable technique, as will be known to those of ordinary skill in the art. In some cases, the aerosol compositions can be prepared by mixing an appropriate amount of a liquefied gaseous propellant and a minoxidil composition under pressure, followed by packing the mixture in an aerosol container. In other cases, an aerosol container can be loaded with a minoxidil composition (e.g., as a powder, slurry, or liquid), followed by pressurizing the container with a propellant and sealing the container.

An aerosol container can be formed of any suitable material, for example, metal (e.g., aluminum), glass, plastic, or combinations thereof. In most embodiments, the aerosol container is formed essentially of metal. The container can comprise a dip tube and/or a spray nozzle. The nozzle on the container must be selected to minimize or avoid clogging, such as may occur with formulations containing starch. Preferably, the aerosol container is a metered dose container such that a fixed amount of the composition is delivered with each activation of the nozzle. For the foam composition, a metered dose may be administered by filling a dosage cup with the foam and then administering the measured dose to the scalp. In another embodiment, the aerosol composition (nonfoaming) is delivered by a continuous valve and the patient or user is instructed on how long to press on the valve to open the nozzle and thereby measure the amount of composition administered.

In one embodiment, the aerosol container is a non-combustible propellant delivery system. In one embodiment, the non-combustible propellant delivery system is a bag-on-valve system.

In an embodiment, the propellant delivery system comprises a portable container having a gas impervious container wall, a pressure source disposed within the container and a selectively operable valve assembly affixed to the container, as described in U.S. Patent Application Publication No. 2012/0259298 to Cronk. The dry shampoo is in liquid form and is disposed within the container. The valve assembly produces a metered spray of the dry shampoo within about 5 seconds following a single operation of said valve assembly. The contents of U.S. Patent Application Publication No. 2012/0259298 to Cronk are fully incorporated herein as if fully presented in this application.

In an embodiment, the propellant delivery system comprises a metered dose dispensing aerosol valve for a container comprising a valve body defining an aperture, a seal mounted at the aperture, a metering chamber, a tank seal, a transfer passage through which a quantity of dry shampoo to be dispensed can pass from the container into the metering chamber and a valve stem having a dispensing passage, the valve stem being slideably moveable through the seal such that in a first position the dispensing passage is isolated from the metering chamber and the metering chamber is in communication with the container via the transfer passage and in a second position the dispensing passage is in communication with the metering chamber to allow dry shampoo to be dispensed from the metering chamber through the dispensing passage and the transfer passage is isolated from the metering chamber, and the valve stem extending, at least in the second position, through the tank seal, wherein the transfer passage comprises a mechanical flow valve positioned towards the end of the transfer passage adjacent the metering chamber, which in said first position, allows the dry shampoo to be dispensed to pass from the container into the metering chamber but not out of the metering chamber, as described in U.S. Pat. No. 6,454,140 to Jinks. The contents of U.S. Pat. No. 6,454,140 to Jinks are fully incorporated herein as if fully presented in this application.

In an embodiment, the propellant delivery system comprises a metered dose dispensing valve for dispensing metered volumes of a pressurized aerosol formulation, said valve comprising a metering chamber, a valve stem extending through the metering chamber, and a movable agitator within said metering chamber, wherein the movable agitator is configured so as to encircle the valve stem within the metering chamber, as described in U.S. Pat. No. 5,593,069 to Jinks The pressurized aerosol formulation comprises a dry shampoo and a propellant. The contents of U.S. Pat. No. 5,593,069 to Jinks are fully incorporated as if fully presented in this application.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

As used herein and in the claims, the singular forms include the plural reference and vice versa unless the context clearly indicates otherwise.

### Definitions

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims.

As used herein the term “comprising” or “comprises” is used in reference to compositions, methods, and respective component(s) thereof, that are useful to an embodiment, yet open to the inclusion of unspecified elements, whether useful or not.

As used herein the term “consisting essentially of ” refers to those elements required for a given embodiment. The term permits the presence of elements that do not materially affect the basic and novel or functional characteristic(s) of that embodiment of the invention.

As used herein, unless stated otherwise, “% by weight” refers to the % weight of the components of the composition exclusive of the propellant. For embodiments describing the weight of the propellant or of a dry shampoo relative to the propellant, the % by weight refers to the % weight by weight of the entire composition.

As used herein, the term “lower alcohol” refers to any straight-chain or branched, saturated or unsaturated, alcohol molecule with 1-10 carbon atoms. In one embodiment, the lower alcohol is ethanol. In one embodiment, the lower alcohol is specifically denatured alcohol available as SDA 40-B or SDA 40-2. In another embodiment, the lower alcohol is ethanol. In one embodiment, the lower alcohol is specifically denatured alcohol available under the name SDA-40-2, which comprises 99.86% (v/v) ethanol (190 or 200 proof) and 0.14% (v/v) tert-butyl alcohol and brucine sulfate.

As used herein, the term “substantially free of water” refers to a water content of no more than 5%, no more than 4%, no more than 3%, no more than 2%, 5%-2%, 4%-2% or no more than 1% by weight in a composition.

As used herein, a weight concentration of “minoxidil or a pharmaceutically acceptable salt thereof” refers to the weight concentration of the minoxidil. For a pharmaceutically acceptable salt of minoxidil, the concentration refers to the weight of the minoxidil in solution.

As used herein, the terms “treat” or “treatment” or “treating” refers to both therapeutic treatment and prophylactic or preventative measures, wherein the object is to prevent, slow, or even reverse the development of hair loss, such as slow down the development of hair loss, or reducing at least one effect or symptom of hair loss. Treatment is generally “effective” if one or more symptoms or clinical markers are reduced. Alternatively, treatment is “effective” if the progression of hair loss is reduced or halted. In some cases, treatment is “effective” if the density of hair is greater than that in absence of the treatment by at least 10%, at least 20%, at least 30%, at least 40%, at least 50%, at least 60%, at least 70%, at least 80%, or at least 90%.

Other than in the operating examples, or where otherwise indicated, all numbers expressing quantities of ingredients or reaction conditions used herein should be understood as modified in all instances by the term “about.” The term “about” when used in connection with percentages may mean ±5% of the value being referred to. For example, about 100 means from 95 to 105.

Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. Further, to the extent not already indicated, it will be understood by those of ordinary skill in the art that any one of the various embodiments herein described and illustrated can be further modified to incorporate features shown in any of the other embodiments disclosed herein.

All patents and other publications; including literature references, issued patents, published patent applications, and co-pending patent applications; cited throughout this application are expressly incorporated herein by reference.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

What is claimed is:

1. An aerosol composition comprising:
a dry shampoo and a propellant,
wherein the dry shampoo comprises:
minoxidil or a pharmaceutically acceptable salt thereof at a concentration of about 2 to about 20% by weight;
a carrier material; and
a modified starch;
wherein the composition is not formulated for application as a foam or mousse.

2. The composition of claim 1, wherein the modified starch is aluminum starch octenyl succinate.

3. The composition of claim 2, wherein the dry shampoo comprises from 2% to 20% by weight of aluminum starch octenyl succinate.

4. The composition of claim 3, wherein the dry shampoo further comprises magnesium stearate, tapioca starch, acrylates copolymer, and/or magnesium carbonate, and wherein the apparent pH of the dry shampoo is from 5.0 to 7.0.

5. The composition of claim 1, wherein the dry shampoo comprises from about 2% to about 5% by weight of minoxidil.

6. The composition of claim 5, wherein the modified starch is aluminum starch octenyl succinate that is present at from 2% to 20% by weight of the dry shampoo.

7. The composition of claim 1, wherein the dry shampoo comprises from about 6% to about 20% by weight of minoxidil.

8. The composition of claim 7, wherein the dry shampoo further comprises an acid selected from the group consisting of hydrochloric acid, sulphuric acid, nitric acid, phosphoric acid, citric acid, acetic acid, lactic acid, succinic acid, and maleic acid, and mixtures thereof.

9. The composition of claim 1 further comprising a pharmaceutically acceptable solvent.

10. The composition of claim 1, wherein the carrier material is selected from the group consisting of lower alcohols, volatile polydimethylsiloxanes, cyclomethicone, cyclohexane siloxane, decamethyltetrasiloxane, octamethyltrisiloxane, decamethylpentasiloxane, decamethylcyclopentasiloxane, octamethylcyclotetrasiloxane, trimethylsilylamodimethicone, phenyl trimethicone, hexamethyidisiloxane, and dimethylsiloxane/methylalkylsiloxane, and combinations thereof.

11. A method of inducing hair growth or reducing the rate of hair loss of a subject in need thereof, comprising applying a therapeutically effective amount of the aerosol composition of claim 1 at least once daily to the scalp of the subject.

12. A liquid composition comprising:
minoxidil or a pharmaceutically acceptable salt thereof at a concentration of about 2 to about 20% by weight;
aluminum starch octenyl succinate at a concentration of from 2% to 20% by weight; and
a pharmaceutically acceptable solvent.

13. The liquid composition of claim 12 comprising from about 2% to about 5% by weight of minoxidil.

14. The liquid composition of claim 12 comprising from 2% to 10% by weight of aluminum starch octenyl succinate.

15. A product comprising a spray device or dropper containing the liquid composition of claim 12.

16. A method of inducing hair growth or reducing the rate of hair loss of a subject in need thereof, comprising applying a therapeutically effective amount of the liquid composition of claim 12 at least once daily to the scalp of the subject.
